(19) United States
(12) Patent Application Publication
(10) Pub. No.: US 2010/0190227 A1
(43) Pub. Date: Jul. 29, 2010

(54) PHOTOREACTOR

(75) Inventors: Jochen Dauth, Burghausen (DE); Harald Voit, Reischach (DE)

Correspondence Address: RATNERPRESTIA, P.O. BOX 980, VALLEY FORGE, PA, 19482, US

(73) Assignee: Wacker Chemie AG, Munich (DE)

(21) Appl. No.: 12/602,424

(22) PCT Filed: May 30, 2008
(86) PCT No.: PCT/EP2008/056666

§ 371 Date: Jan. 18, 2010

(30) Foreign Application Priority Data
Jun. 1, 2007 (DE) 10 2007 025 748.3

(51) Int. Cl. B01D 53/62; C12P 3/00; C12N 1/12; C12M 1/42; B01J 19/08

(52) U.S. Cl. 435/168; 423/220; 435/257.1; 435/292.1; 422/186

(57) ABSTRACT

The invention relates to photoreactors (1) comprising LED plastic moulded parts (6) in which at least one LED luminous body (7) is incorporated into a plastic matrix, as a radiation source arranged inside the photoreactor.

### CROSS REFERENCE TO RELATED APPLICATIONS

This is a U.S. National Phase application of PCT application number PCT/EP2008/056666, filed May 30, 2008, which claims priority benefit of German application number DE 10 2007 025 748.3 (filed Jun. 1, 2007), the content of such applications being incorporated herein by reference.

### FIELD OF THE INVENTION

The invention relates to a photoreactor comprising LED plastic molded parts, preferably LED silicone molded parts, optionally in combination with light-guide molded parts as a radiation source; in particular photobioreactors comprising LED plastic molded parts, preferably LED silicone molded parts, optionally in combination with light-guide molded parts as a radiation source.

### BACKGROUND OF THE INVENTION

Photoreactors, in particular photobioreactors, are used for the industrial production of microalgae, for example spirulina, chlorella, chlamydomonas or haematococcus, photosynthetic bacteria such as for example cyanobacteria (for example rhodobacter, rhodospirillum), mosses or other plant cell cultures. Such microalgae and cyanobacteria are capable of converting CO₂ and water into biomass with the aid of light energy (photosynthesis). Two pigment collectives are generally involved in this, the first pigment collective having a good light absorption at wavelengths of about 450 nm, and the second pigment collective having good light absorption at a wavelength of about 680 nm. The formation of biomass takes place in a light reaction and a dark reaction. The light reaction serves to convert radiation energy into chemical energy or light quanta by forming oxygen (=photochemical water cleavage). Formation of the biomass (CH₂O)ₙ takes place in the 2^{nd} step by consuming the light quanta (dark reaction).

Photobioreactors are used for the production of alga biomass and for example foodstuffs, food supplements, proteins, lipids, vitamins, antioxidants, active agents for pharmaceuticals and cosmetics, as well as oil from algae. CO₂ as a carbon source for alga cultivation may be supplied to the photobioreactor as air, air enriched with CO₂, waste gas containing CO₂ or pure CO₂. Such photobioreactors can also be used for CO₂ removal from waste gases.

First-generation photobioreactors use sunlight as a light source. The reactors consist of large open tank systems, for example round tank systems with diameters of up to 45 m and revolving mixer arms. A disadvantage with this is the dependency on the intensity of the solar irradiation and the introduction of contamination owing to the open system (www.ybsweb.co.jp: YAEYAMA premium quality chlorella).

The first photobioreactor system in the world for producing microalgae in closed sterilizable reactors was put into operation by Bisantech in 2000 (www.bisantech.de). Here again, the dependency on the intensity of the solar irradiation is to be mentioned as a disadvantage.

A closed photobioreactor with artificial illumination is known from U.S. Pat. No. 6,602,703 B2. Here, fluorescent tubes arranged parallel in the reactor are used as a light source. A disadvantage here is the relatively high energy demand and the susceptibility of the illumination device to fouling. US Patent Application US 2006/0035370 A1 describes a multistage photobioreactor for the growth-coupled production of a useful metabolite, consisting of a first cultivation zone which contains the microorganisms and a culture for the vegetative growth, and a second cultivation zone which lies close to the first zone on one side and contains a culture medium and microorganisms for the production of the metabolite. The two culture regions are separated from one another by a transparent partition wall. Sunlight or artificial light is used as the light source, with the disadvantages already mentioned above.

It is known from WO 92/00380 A1 to illuminate a photobioreactor by installing a light source outside the reactor and transporting its light into the reactor through light guides. The use of LED luminous bodies (LED=light-emitting diode) as a radiation source for photobioreactors is known: US 2005/0135104 A1 describes the use of LEDs for the illumination of culture containers for marine cultures. The LEDs are enclosed in a transparent housing. The embedding of LEDs in a transparent plastic matrix is not described. From JP 2007-040176 A, it is known to provide the electricity supply for artificial illumination, for example by means of LEDs, of reactors for alga cultivation by means of wind power plants. JP 2000-325073 A describes a container divided into two for alga cultivation. The two compartments of the container are separated by a structure which comprises a circuit board equipped with LEDs. This LED-equipped circuit board is separated from the culture medium on both sides by means of transparent plates. JP 10-098964 A and JP 11-089555 A describe alga reactors in which LEDs are used for illumination. In one embodiment, LED chains are introduced into a transparent tube and this is lowered for illumination in the reactor. In another embodiment, the LED chain is initially welded onto a substrate and then introduced into a transparent tube. JP 2002-315569 A describes a method for alga production, LEDs being used for illumination. To this end, long LED chains are either placed between acrylic glass plates or suspended in transparent tubes. The use of LEDs as a light source for bioreactors in a closed reactor is described in WO 2007/047805 A2. A disadvantage with this is that the illumination is carried out by means of externally placed LEDs. The radiation through the medium is therefore insufficient, allows only short light path lengths of a few centimeters and requires a large surface area for mass production.

It was therefore an object to provide a photoreactor which allows cultivation in a closed system and with a maximally uniform and optimal radiation intensity throughout the volume. It was another object to increase the productivity of photosynthesis reactors, in order to optimize production on an industrial scale. The LEDs should furthermore be provided in the form of molded parts such as can effectively withstand a wide variety of environmental effects, whether physical, chemical or biological, over a long period of time. It was another object to effectively protect not only the individual LEDs but also the electrical links between the LEDs and their connections to electricity sources, against such environmental effects.

### SUMMARY OF THE INVENTION

The invention relates to photoreactors comprising LED plastic molded parts in which one or more LED luminous bodies are enclosed in a plastic matrix, as radiation sources which are arranged inside the photoreactor.

### BRIEF DESCRIPTION OF THE DRAWINGS

FIG. 1 shows a photobioreactor element with LED silicone molded parts in a tube bundle or pipe bundle, according to the invention.

FIG. 2 shows a bubble column photobioreactor, according to the invention.

FIG. 3 shows a bubble column photoreactor with LED silicone tubes connected in parallel, according to the invention.

### DETAILED DESCRIPTION OF THE INVENTION

Closed reactors are preferred as photoreactors. The reactors are manufactured with a pressure-bearing, optionally heat-resistant shell, for example of steel, stainless steel, plastic, enamel or ceramic. Transparent or non-transparent materials may be used, non-transparent materials being preferred for closed reactors. Any desired reactor volumes may be chosen. In contrast to the prior art, restriction to small volumes for closed reactors is not necessary since uniform illumination of the interior can be obtained with the internally arranged LED plastic molded parts as radiation sources, so that the reactor dimensions can be decoupled from the light path.

In contrast to reactors which consist of glass or transparent plastic, the pressure-bearing materials allow a tall design, with a reduced footprint, for mass production. Arranging the LED plastic molded parts as lighting inserts in such reactors makes it possible to combine a plurality of reactor segments to form tall columnar reactors with a corresponding space saving, that is to say an enormously high productivity in relation to the footprint. It is furthermore possible to operate with elevated pressure, preferably with a gauge pressure in the range of from 0.1 to 5 bar. In photobioreactors, a higher pressure entails a higher CO₂ partial pressure in the gas containing CO₂, with which the reactor is provided, which leads to a higher CO₂ equilibrium concentration in the medium, accelerates the CO₂ transport into the microalgae and makes the photosynthesis reaction more intensive. If waste gas containing CO₂ is used as the CO₂ source, the equilibrium concentration in the medium is likewise/additionally increased and photosynthesis and alga growth are therefore accelerated considerably.

The tall design of the photobioreactor, with a reduced footprint, creates a longer gas residence time (the rate of ascent of the gas bubbles remains the same) and therefore achieves much better CO₂ depletion. Besides the better CO₂ depletion, greater enrichment of gases in the waste gas of the photobioreactor can also be obtained with such systems, so that for example it is possible to obtain oxygen or even hydrogen, in the case of algae which produce hydrogen, in an economically viable fashion. A closed reactor furthermore reduces or prevents evaporation loss of water, which is advantageous particularly in regions of the earth which are short of water (arid regions).

When using LED plastic molded parts, optionally in combination with light-guide molded parts, as radiation sources which are arranged inside the photoreactor, there is furthermore no longer any dependency on sunlight.

The pressure- and heat-resistant design of the reactor shell, in conjunction with heat-resistant LED plastic molded parts, also makes it possible optionally to operate at elevated temperature. It is possible to sterilize the reactor with steam at 120° C. and therefore effectively avoid contamination, and also to use genetically modified microalgae or cyanobacteria. The reactor can likewise be cleaned very effectively by CIP (“cleaning in place”) steam jets at 120° C.

The reactor is equipped with supply lines for filling and nutrient supply, and with discharge lines for product extraction and emptying. For continuous operation, it may be recommendable to equip it with an external link in which phase separation apparatus or modules for dialysis, reverse osmosis and micro- or nanofiltration are arranged. For thermal dissipation and heating, the reactor may optionally be equipped with a double shell, semi-tube coils on the reactor walls or internally placed heat exchangers. The reactor may furthermore also contain stirring devices and pumps for mixing. The mixing is preferably carried out by gassing with the gas in a similar way to a bubble column according to the airlift principle, without additional mechanical energy. Bubble columns/airlift reactors are operated with a gas flow rate of preferably from 0.005 to 2.0 m/s, particularly preferably in a range of from 0.02 to 0.2 m/s. The reactor is preferably subdivided into a plurality of reactor units, with the reactor units arranged stacked on one another preferably with an offset, the reactor units being connected to one another through access openings. The reactor cells may be connected to one another by flanges (in a similar way to column trays) or preferably formed by inserts (in a similar way to column bases) in a common outer shell.

During operation as a photobioreactor, for example with an alga growth rate of 0.025 l/h, an alga concentration of 2.5 wt % in the aqueous reactor medium, and a gas flow rate of 0.05 m/s, when using a typical power plant waste gas as the CO₂ source, the optimal reactor height is about 20 m.

LED plastic parts, which contain one or more LED luminous bodies in one or more plastic matrices, preferably LED silicone molded parts, are used as the radiation source. Suitable LED luminous bodies are radiation-emitting semiconductor components comprising organic or inorganic semiconductors, so-called LEDs. The LEDs may be diodes already encapsulated with plastic, usually silicone, or unencapsulated diodes. The LEDs may radiate in the infrared range, in the visible range or in the UV range. The choice depends on the intended applications. For photosynthesis in photobioreactors, LEDs which radiate in the visible range, in particular red light, are preferred. The LED luminous bodies embedded in the plastic matrix may emit at the same wavelength. Nevertheless, LED luminous bodies with different radiation characteristics may also be combined with one another. In general, a plurality of LED luminous bodies are joined together conductively and connected in series and/or parallel. The luminous body arrangement may be connected to sensors, as well as to measurement/control devices. The number of luminous bodies and their mutual arrangement depend on their application. The LED luminous bodies may be operated continuously or in pulses. Any desired electricity supply may be used, although it is preferable to employ solar cells.

A light guide is a fiber of a transparent material that transmits light, usually glass or plastic, which is used to transport light or infrared radiation. Examples of light guides are optical waveguides, glass fibers, polymer optical fibers or other light-guiding components made of plastic, as well as fiber-optic components. The light guides are organized so that the light is emitted uniformly over the entire extent of the light guide. Optionally, the light guide may be equipped with a lens in order to concentrate and amplify the light before introduction into the light guide. The use of light-guide molded parts as a radiation source besides plastic LED molded parts is advantageous for operation during the day. Light guides based on thermoplastic silicone elastomers (TPSE) are preferred. Thermoplastic silicone elastomers contain an organopolymer component, for example a polyurethane or a polyvinylester, and a silicone component, usually based on a polydialkylsiloxane basis with the specification mentioned above. Suitable thermoplastic silicone elastomers are available on the market, for example the corresponding Geniomer^{R} types from Wacker Chemie.

Any desired geometrical configuration of the LED plastic molded parts and the light guides may be used. They may be provided in the form of pipes, as strips, as tubes, as plates or in the form of mats.

Plates, mats or strips, particularly in the case of LED plastic molded parts as the radiation source, are preferably used in order to equip the inner walls of the reactor with radiation sources. Configuration in the form of tubes or pipes is recommendable for the installation of radiation sources inside the reactor.

For uniform light supply inside large reactors, a plurality of LED plastic molded parts, around which the alga suspension flows and which are designed for example as tubes, pipes or plates, may be combined as tube, pipe or plate bundles to form lighting inserts. In a particularly preferred embodiment, the luminous bodies may be arranged mutually offset (for example helically) in such a way that they illuminate the surrounding space fully and homogeneously.

In the case of a tube bundle consisting of LED plastic molded parts, the required distance between neighboring molded part surfaces and light emission approximately corresponding to natural solar irradiation, relative to the molded part surface, is about 1 to 10 cm, preferably from 2 to 5 cm.

A photobioreactor may also contain LED plastic molded parts in the form of tubes or plates, through which the alga suspension flows. A plurality of tubes or plates may in this case be combined to form tube or plate bundles, and form a reactor unit through which a cooling medium flows in the shell space and which is thereby cooled. Any desired dimensioning of the LED plastic molded parts and the light-guide molded parts may be used, and may be adapted to the reactor size.

The distance between the LED plastic molded parts and/or the light-guide molded parts may be increased by supportive light distribution inside the photoreactor using “luminescent silicone floating bodies” with an adjusted optimal wavelength and a minimal density difference, which are preferably suspended merely by the gassing. These “luminescent silicone floating bodies” consist of one or more luminescent substances in a silicone matrix, for example of thermoplastic silicone elastomers, as described in EP 1412416 B1 or EP 1489129 B1.

Suitable materials for the LED plastic molded parts are thermoplastics and thermosetting plastics such as acrylic glass, polyethylene, polypropylene, PVC, polyamides, polyesters such as PET. Silicones are preferred.

In the LED plastic molded parts, the LEDs are fully embedded in the plastic matrix and encapsulated by it, preferably including the electrically conductive links between the LEDs, and particularly preferably also the connections of the LED chains to the electricity supply.

Examples of suitable silicones are crosslinked silicone rubbers which undergo radical crosslinking by a condensation or addition reaction. The crosslinking reaction may be initiated cationically, by means of appropriate catalysts, or radically, by means of peroxides, or by radiation, in particular UV radiation, or thermally. Systems which lead to crosslinked silicone rubbers are preferably available on the market as 1- or 2-, but also as multi-component systems. Silicone hybrid polymers and silicone resins are also suitable. The layer thickness of the silicone matrix depends on the application of the LED silicone molded parts, and is generally between 0.1 and 50 mm.

Condensation-crosslinking silicone rubber systems contain

a) organopolysiloxanes having condensable end groups
b) optionally organosilicon compounds having at least three hydrolyzable groups bound to silicon per molecule, and
c) condensation catalysts.

Suitable crosslinked silicone rubbers, which crosslink by a condensation reaction, are 1-component systems which crosslink at room temperature, so-called RTC-1 silicone rubbers. RTC-1 silicone rubbers are organopolysiloxanes with condensable end groups, which crosslink in the presence of catalysts by condensation at room temperature. The most common are dialkylpolysiloxanes of the structure R₃SiO[—SiR₂O]ₙ—SiR₃ with a chain length of n>2. The alkyl radicals R may be identical or different and generally have from 1 to 4 C atoms, and may optionally be substituted. The alkyl radicals R may also be partially replaced by other radicals, preferably by aryl radicals which are optionally substituted, the alkyl (aryl) groups R being partially replaced by condensation-crosslinkable groups, for example alcohol, acetate, amine or oxime radicals. The crosslinking is catalyzed by means of suitable catalysts, for example tin or titanium catalysts.

Suitable RTC-1 silicone rubbers are available on the market, for example the corresponding types of the ELASTOSIL® A, E or N series from Wacker Chemie AG. Suitable crosslinked silicone rubbers, which crosslink by a condensation reaction, are 2-component systems which crosslink at room temperature, so-called RTC-2 silicone rubbers. RTC-2 silicone rubbers can be obtained by means of condensation-crosslinking organopolysiloxanes, multiply substituted with hydroxyl groups, in the presence of silicic acid esters. Also usable as crosslinkers are alkyl silanes with alkoxy, oxime, amine or acetate groups, which crosslink in the presence of suitable condensation catalysts, for example tin or titanium catalysts with the polydialkylsiloxanes terminated by hydroxyl groups. Suitable RTC-2 silicone rubbers are available on the market, for example the corresponding types of the ELASTOSIL® RT series from Wacker Chemie AG.

Examples of the polydialkylsiloxanes contained in RTC-1 and RTC-2 silicone rubber are those of the formula (OH)R₂SiO[—SiR₂O]—SiR₂(OH) with a chain length of n>2, in which case the alkyl radicals R may be identical or different, generally contain from 1 to 4 C atoms and may optionally be substituted. The alkyl radicals R may also be partially replaced by other radicals, preferably by aryl radicals which are optionally substituted. The polydialkylsiloxanes preferably contain terminal OH groups which crosslink with the silicic acid esters or the system alkylsilane/tin (titanium) catalyst at room temperature.

Examples of the alkylsilane, having hydrolyzable groups, contained in RTC-1 and RTC-2 silicone rubbers are those of the formula RₐSi(OX)₄₋ₐ, with a=1 to 3 (preferably 1) and X denoting R″ (alkoxy crosslinker), C(O)R″ (acetate crosslinker), N═CR″₂ (oxime crosslinker) or NR″₂ (amine crosslinker), where R″ denotes a monovalent hydrocarbon radical having from 1 to 6 carbon atoms.

Addition-crosslinking silicone rubber systems contain

a) organosilicon compounds which have radicals with aliphatic carbon-carbon multiple bonds,
b) optionally organosilicon compounds having Si-bound hydrogen atoms or, instead of a) and b),
c) organosilicon compounds which have radicals with aliphatic carbon-carbon multiple bonds and Si-bound hydrogen atoms
d) the attachment of Si-bound hydrogen atoms to catalysts which promote aliphatic multiple bonding and
e) optionally the attachment of Si-bound hydrogen atoms to catalysts which retard aliphatic multiple bonding at room temperature.

Suitable crosslinked silicone rubbers, which crosslink by an addition reaction, are 2-component systems which crosslink at room temperature, so-called RTC-2 silicone rubbers. Addition-crosslinking RTC-2 silicone rubbers are obtained by the crosslinking, catalyzed by Pt catalysts, of multiply ethylenically unsaturated groups, preferably vinyl groups, of substituted organopolysiloxanes with organopolysiloxanes multiply substituted with Si—H groups in the presence of platinum catalysts.

One of the components preferably consists of dialkylpolysiloxanes having the structure R₃SiO[—SiR₂O]ₙ—SiR₃ with n≧0, generally having from 1 to 4 C atoms in the alkyl radical, in which case the alkyl radicals may be fully or partially replaced by aryl radicals such as the phenyl radical, one of the terminal radicals R at one or both ends being replaced by a polymerizable group such as the vinyl group. Radicals R in the siloxane chain may likewise partially be replaced by polymerizable groups, optionally in combination with the radicals R of the end groups. Vinyl end-blocked polydimethylsiloxanes of the structure CH₂═CH₂—R₂SiO[—SiR₂O]ₙ—SiR₂—CH₂═CH₂ are preferably used.

The second component contains an Si—H functional crosslinker. The polyalkylhydrogensiloxanes conventionally used are copolymers of dialkylpolysiloxanes and polyalkylhydrogensiloxanes having the general formula R₁₃SiO[—SiR₂O]ₙ—[SiHRO]ₘ—SiR′₃ with m≧0, n≧0 and the proviso that they must contain at least two Si—H groups, where R′ can denote H or R. There are therefore crosslinkers with lateral and terminal Si—H groups, while siloxanes with R′═H, which only have terminal Si—H groups, may also be used as well for chain lengthening.

They contain small amounts of platinum-organic compounds as crosslinking catalysts.

Suitable RTC silicone rubbers are available on the market, for example the corresponding types of the ELASTOSIL® RT or ELASTOSIL® LR (LSR silicone rubber) or SEMICOSIL® series from Wacker Chemie AG.

Suitable silicone rubbers which crosslink radically or by an addition reaction are solid silicone rubbers which crosslink with temperature elevation (HTC).

Addition-crosslinking HTC silicone rubbers are obtained by the crosslinking of multiply ethylenically unsaturated groups, preferably vinyl groups, of substituted organopolysiloxanes with organopolysiloxanes multiply substituted with Si—H groups in the presence of platinum catalysts.

One of the components of the HTC silicone rubbers which crosslink peroxidically or by addition preferably consists of dialkylpolysiloxanes having the structure R₂SiO[—SiR₂O]ₙ—SiR₃ with n≧0, generally having from 1 to 4 C atoms in the alkyl radical, in which case the alkyl radicals may be fully or partially replaced by aryl radicals such as the phenyl radical, one of the terminal radicals R at one or both ends being replaced by a polymerizable group such as the vinyl group. Polymers with lateral or lateral and terminal vinyl groups may however also be used. Vinyl end-blocked polydimethylsiloxanes of the structure CH₂═CH₂—R₂SiO[—SiR₂O]ₙ—SiR₂—CH₂═CH₂ are preferably used, as well as vinyl end-blocked polydimethylsiloxanes of said structure, which also carry lateral vinyl groups. In the case of addition-crosslinking HTC silicone rubbers, the second component is a copolymer of dialkylpolysiloxanes and polyalkylhydrogensiloxanes having the general formula R′₃SiO[—SiR₂O]ₙ—[SiHRO]ₘ—SiR₃ with m≧0, n≧0 and the proviso that they must contain at least two Si—H groups, where R′ can denote H or R. There are therefore crosslinkers with lateral and terminal Si—H groups, while siloxanes with R′═H, which only have terminal Si—H groups, may also be used as well for chain lengthening. Platinum catalysts are used as crosslinking catalysts.

HTC silicone rubbers are also processed as single-component systems, the crosslinking reaction being initiated by temperature elevation and in the presence of peroxides as crosslinking catalysts, for example acyl, alkyl or aryl peroxides. Peroxide-crosslinking HTC silicone rubbers are obtained by the crosslinking of organopolysiloxanes optionally multiply substituted with ethylenically unsaturated groups, preferably vinyl groups. Suitable HTC silicone rubbers are available on the market, for example the corresponding ELASTOSIL® R or ELASTOSIL® R plus types from Wacker Chemie AG.

Recently, special HTC and RTC-1 silicone rubbers have also become available on the market, which are crosslinked via the described addition reaction by special platinum complexes or platinum/inhibitor systems being thermally and/or photochemically activated and therefore catalyzing the crosslinking reaction. Such systems are available, for example, as ELASTOSIL® R types, ELASTOSIL® RT types and Semicosil® types from Wacker Chemie AG.

Suitable materials are also silicone hybrid polymers. Silicone hybrid polymers are copolymers or graft copolymers of organopolymer blocks, for example polyurethane, polyurea or polyvinyl esters, and silicone blocks generally based on polydialkylsiloxanes with the specification mentioned above. For example, thermoplastic silicone hybrid polymers are described in EP 1412416 B1 and EP 1489129 B1, the disclosure of which in this regard is also intended to be included in the subject-matter of this application. Such silicone hybrid polymers are referred to as thermoplastic silicone elastomers (TPSE) and are available on the market, for example the corresponding GENIOMER® types from Wacker Chemie AG.

Silicone resins are likewise suitable materials for the silicone matrix. In general, the silicone resins contain units of the general formula R^{b}(RO)_{c}SiO_{(4-b-c)/2}, where

b is equal to 0, 1, 2 or 3,
c is equal to 0, 1, 2 or 3,
with the proviso that b+c 3,
and R has the meaning indicated for it above,
which form a highly crosslinked organosilicon network. Suitable silicone resins are available on the market, for example the corresponding SILRES® types from Wacker Chemie AG.

Radiation-curing acrylic-, epoxy- or vinyl ether-functional silicones are also suitable, which are cured by radical formers or cationic photoinitiators.

If the LED luminous bodies are embedded in a single silicone matrix, then the silicone matrix must be bonded fully and permanently to the LED luminous body throughout the operating time of the reactor, so that the luminous power does not decrease over the operating time of the photoreactor. Silicones which match their shape to the luminous bodies, adhere well and do not form any cavities between the matrix and the luminous body, for example owing to temperature variations which occur, are particularly suitable for this. Preferred materials are said RTC-2 silicone rubbers, in particular LSR silicone rubber, HTC silicone rubber and silicone hybrid polymers, in particular thermoplastic elastomers such as those described above.

In a preferred embodiment, the LED silicone molded parts contain a soft inner silicone matrix A, which is enclosed by one or more harder silicone matrices B. The inner silicone matrix A is soft with a Shore A hardness (DIN 53 505/ISO 868) of less than or equal to 10 or, if it is a liquid silicone oil, an average viscosity (at 23° C. and 1013 mbar) of from 1 to 100×10⁶ mPa·s. Preferably, the Shore A hardness is less than 5 or the average viscosity (at 23° C. and 1013 mbar) is from 10×10¹⁶ mPa·s. For the outer silicone matrix B, the Shore A hardness is more than 10 and, in the event that the inner silicone matrix A likewise consists of a type of silicone which is solid at standard conditions (23/50 DIN 50014), the difference between the Shore A hardnesses of the inner silicone matrix A and the outer silicone matrix B is at least 5 Shore hardness points, preferably at least 10, in particular at least 20.

The outer silicone matrix B is based on the materials mentioned above.

The silicone matrix A is optimized for the luminous body and, besides the protective function for the electronic components (shock absorption), should optimize the luminous efficiency (refractive index matching) and facilitate thermal dissipation. It is also important for the luminous body to be firmly enclosed by the silicone matrix throughout the operating time of the luminous body, and to prevent air and water inclusions which lead to diffuse light scattering effects.

Preferred materials for the inner silicone matrix A are silicone oils, which are generally dialkylpolysiloxanes of the structure R₃SiO[—SiR₂O]ₙ—SiR₃ with a chain length of n>2. The alkyl radicals R may be identical or different and generally have from 1 to 4 C atoms, and may optionally be substituted. The alkyl radicals R may also be partially replaced by other radicals, preferably by aryl radicals which are optionally substituted, or by trialkylsiloxy groups in the case of branched silicone oils. Examples are methyl silicone oils (CH₃)₃SiO[—Si(CH₃)₂O]ₙ—Si(CH₃)₃, methyl phenyl silicone oils (CH₃)₃SiO [—Si (CH₃)₂O]_{n′}—[—Si (C₆H₅)₂O]_{n″}—Si (CH₃)₃ or (CH₃)₃SiO[—Si(CH₃)₂O]_{n′}—[—Si(CH₃)(C₆H₅)O]_{n″}—Si (CH₃)₃, in both cases with n′+n″>2, branched methyl silicone oils (CH₃)₃SiO[—Si(CH₃)(OSi(CH₃)₃)O]ₙ—Si(CH₃)₃, branched methyl phenyl silicone oils (CH₃)₃SiO[—Si(C₆H₅)(OSi(CH₃)₃)O]ₙ—Si(CH₃)₃. By introducing aryl groups and adjusting the ratio of alkyl to aryl groups, the person skilled in the art can match the refractive index of the silicone matrix to the luminous body in a known way. Furthermore, polydimethylsiloxane oils functionalized (and “non-stoppered”) on the end groups may also preferably be used. Such silicone oils are available on the market and can be produced by known methods. Examples of commercially available silicone oils are the Wacker silicone oils from Wacker Chemie AG.

Silicone gels are also suitable for the inner silicone matrix A. Silicone gels are produced from two pourable components, which crosslink at room temperature in the presence of a catalyst. One of the components generally consists of dialkylpolysiloxanes having the structure R₃SiO[—SiR₂O]ₙ—SiR₃ with n≧0, generally having from 1 to 4 C atoms in the alkyl radical, in which case the alkyl radicals may be fully or partially replaced by aryl radicals such as the phenyl radical, one of the terminal radicals R at one or both ends being replaced by a polymerizable group such as the vinyl group. Radicals R in the siloxane chain may likewise partially be replaced by polymerizable groups, optionally in combination with the radicals R of the end groups. Vinyl end-blocked polydimethylsiloxanes of the structure CH₂═CH₂—R₂SiO[—SiR₂O]ₙ—SiR₂—CH₂═CH₂ are preferably used.

The second component contains an Si—H functional crosslinker. The polyalkylhydrogensiloxanes conventionally used are copolymers of dialkylpolysiloxanes and polyalkylhydrogensiloxanes having the general formula R′₃SiO[—SiR₂O]ₙ—[SiHRO]ₘ—SiR′₃ with m≧0, n≧0 and the proviso that they must contain at least two Si—H groups, where R′ can denote H or R. There are therefore crosslinkers with lateral and terminal Si—H groups, while siloxanes with R′═H, which only have terminal Si—H groups, may also be used as well for chain lengthening. They contain small amounts of platinum-organic compounds as crosslinking catalysts. By mixing the components, the crosslinking reaction is initiated and the gel is formed. The crosslinking reaction may be accelerated by the effect of heat and/or by electromagnetic radiation, preferably UV radiation. UV LEDs themselves may induce the crosslinking reaction of gels. Silicone gels are particularly soft materials, particularly preferably with a SHORE 00 hardness of less than 50 (DIN 53 505/ISO 868), most preferably those with a penetration value according to DIN ISO 2137 of >10 mm/10 (with a 9.38 g quarter cone and an action time of 5 s). Suitable silicone gels are available on the market, for example under the brand name Wacker SilGel® from Wacker Chemie AG, Munich.

The LED plastic molded parts may be produced by means of techniques which are conventional in plastics processing, for example by means of casting, extrusion, cast molding, compression molding or injection molding, depending on the configuration of the LED plastic molded parts. The LED plastic molded parts may optionally also be covered with a topcoat, for example based on silicone resins.

By an optimized design arrangement of a large number of LED plastic molded parts inside large fermenters, in the scope of reactor inserts, economically viable mass production of alga biomass is made possible in fermenters with large volumes. With the LED plastic molded parts, a multiplicity of LEDs can be combined to form reactor lighting inserts, which therefore economically allow optimal internal illumination of a large reactor volume.

Embedding the LEDs in a plastic matrix, particularly in the case of a silicone matrix, significantly reduces fouling and deposit formation. Another advantage is that the use of LED silicone molded parts allows repeated steam sterilization of the entire photobioreactor at least at 121° C. over a period of at least 1 hour.

Arranging the LED plastic molded parts throughout the reactor volume achieves better thermal dissipation, so that the LEDs can be operated with a higher voltage and luminous efficiency than is possible with an external arrangement.

The photoreactor may be used as a reactor for radiation-induced chemical reactions. The photoreactor may be used as a photobioreactor for the autotrophic and heterotrophic production of valuable substances such as proteins, vitamins, pharmaceutical active agents, lipids, and hydrogen from CO₂, water and mineral nutrients, by means of microalgae. Other applications are the mixotrophic production of valuable substances by feeding organic carbon sources, the production of alga biomass from CO₂ and water and mineral nutrients, and the removal of CO₂ from power plant or industrial waste gases. The alga biomass produced in this way is suitable as an energy raw material, chemical raw material, foodstuff, and for use in cosmetic and medical applications. The reactor is furthermore suitable for methods of producing hydrogen or other gaseous metabolic products using microalgae or microorganisms, which require an energy supply in the form of light.

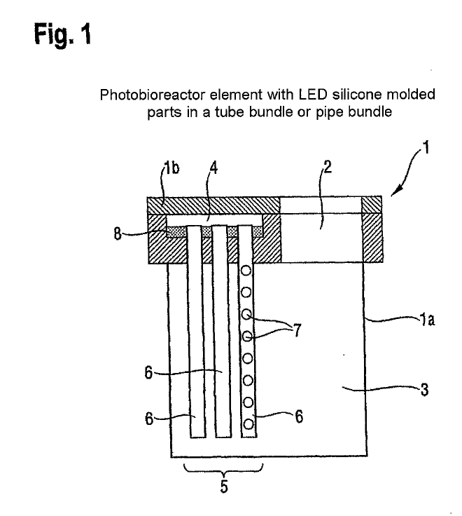

The invention will be explained by way of example in FIGS. 1 to 3:

FIG. 1: Photobioreactor element with LED silicone molded parts in a tube bundle or pipe bundle.

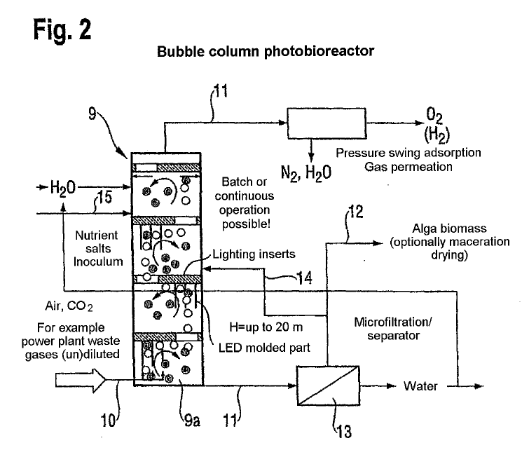

The photobioreactor element 1 is constructed from a pressure-bearing reactor shell 1a and comprises a lighting insert 1b, in the capacity of a column insert (special column base) or a lighting insert flange, which may in a special case also be the photobioreactor top. Connections for supplies and discharges may be arranged in the reactor shell 1a or photobioreactor top 1b. The lighting insert or the lighting insert flange 1b has an access opening 2 for the next photobioreactor element (see FIG. 2). The photobioreactor is filled with a gassed aqueous alga suspension 3 and, through the access opening of the photobioreactor element underneath, is in communication with it in respect of gas and suspension. The gas bubbles ascend and induce liquid circulation, mixing and suspension of the microalgae.

The lighting insert (lighting insert flange, in the special case photobioreactor top) 1b contains a parallel-connected bundle 5 of LED silicone molded parts 6, which contain red LEDs (red points) 7 connected in series. The bundle 5 is connected to the tube bottom of the lighting insert (lighting insert flange, in the special case photobioreactor top) 1b by means of a cast sealing compound.

FIG. 2: Bubble column photobioreactor

FIG. 2 shows a photobioreactor consisting of a plurality of photobioreactor elements according to FIG. 1 assembled to form a column reactor 9. At the lower end of the reactor column 9, air or power plant gas containing CO₂ is fed to the lowermost reactor component 9a through a line 10 and a gas distributor. At the upper end, the CO₂-depleted, purified gas emerges through a line 11, or gaseous products can be removed and separated in subsequent method stages. Aqueous alga suspension can be removed through a line 12. The alga biomass can be concentrated as a retentate in a microfiltration/separation unit 13. A substream can be fed back through a line 14 in order to increase the microalga concentration. The water, optionally after an enrichment with nutrient salts, is preferably supplied at the top into the photobioreactor and, if a microfiltration/separating unit 13 is installed, separated as a permeate. A substream may optionally be fed back through a line 15. The column reactor 9 may be operated in batch mode, in feed batch operation or continuously.

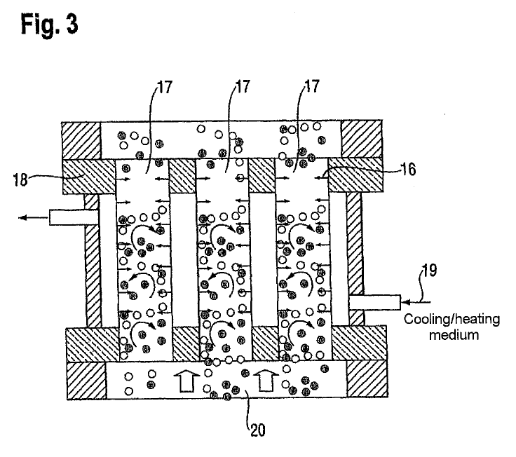

FIG. 3: Bubble column reactor with LED silicone tubes connected in parallel

FIG. 3 shows a photobioreactor in which a plurality of transparent silicone tubes 17 equipped with LEDs 16 are connected in parallel as a tube bundle, and are connected to a frame 18. The silicone tubes are thermally regulated by a heating/cooling system 19. A plurality of such photoreactor inserts may be connected above one another as a column reactor and/or installed in stirred, airlift, bubble column or loop reactors.

### Example 1

A closed photosynthesis tube reactor made of stainless steel with a diameter of 365 mm, a height of 28 m, and having 27 lighting inserts arranged above one another with a respective spacing of 1 m, each with 255 LED silicone molded parts projecting downward (dimensions (H×W×D) 965 mm×8 mm×13 mm) consisting of an LED strip embedded in the silicone (connector rail 8.4 W; Vf=12 V If=700 mA) with a distance of about 2 cm between two LED silicone molded parts, was operated for the autotrophic production of alga biomass (Chlorella vulgaris) under sterile conditions semi-continuously over a period of 4 weeks.

The reactor was configured with externally welded semi-tube loops for sterilization and cooling, and equipped with temperature, pressure, pH and level regulation.

The sterilization was carried out when filled with medium (water+nutrient salts) at 121° C. and with a positive pressure over a period of one hour. To this end, steam was passed at 6 bar through the semi-tube loops, the steam supply being regulated via the temperature in the reactor. During cooling, the gassing was started first in order to avoid the creation of a negative pressure.

The operating temperature in the reactor was subsequently regulated constantly via the cooling water supply in the double shell and adjusted to 27° C. A slight positive pressure of 10 mbar was set up at the reactor head. The air input and output and the water feed were filtered sterilely, and the alga suspension was emptied into a sterilized collection container.

The reactor was seeded with a chlorella pre-culture which was produced in shaking flasks.

After reaching a dry biomass concentration of 2% (20 g/l), continuous operation was adopted.

The dilution rate was adjusted so that an aqueous alga suspension (algae: Chlorella vulgaris) with an alga content of 2 wt % was obtained in the steady state. For the water supply of the algae and the feed of nutrient salts, this required a continuous feed water flow rate of 8.8 l/h. The system was supplied with 20 m³/h [stp] of a waste gas comprising CO₂, which contained 4.5 vol % CO₂, 10.2 vol % O₂, 10.8 vol % H₂O and 74.5 vol % N₂.

The waste gases emerging from the reactor had the composition 0.1% CO₂, 16.0% O₂, 2.9% H₂O and 81.0% N₂. The 2% strength alga suspension was taken from the reactor continuously while regulating the filling level. The reactor was sampled 3× daily. The reactor productivity in continuous permanent operation was 0.4 kg of dry alga mass/m³/h.

1-21. (canceled)

22. A photoreactor comprising LED plastic molded parts in which a plurality of LED luminous bodies are enclosed in a plastic matrix, as radiation sources which are arranged inside the photoreactor, wherein the LED plastic molded parts comprise LED silicone molded parts.

23. The photoreactor as claimed in claim 22, wherein radiation-emitting semiconductor components comprising organic or inorganic semiconductors are used as the LED luminous bodies.

24. The photoreactor as claimed in claim 22, wherein the LED silicone molded parts contain a plurality of LED luminous bodies joined together conductively and connected in series and/or parallel.

25. The photoreactor as claimed in claim 22, further comprising light-guide molded parts as a radiation source.

26. The photoreactor as claimed in claim 25, wherein the light-guide molded parts are based on thermoplastic silicone elastomers.

27. The photoreactor as claimed in claim 22, further comprising luminescent silicone floating bodies for supportive light distribution in the photoreactor.

28. The photoreactor as claimed in claim 25, wherein the LED plastic molded parts and the light guide molded parts are provided in the form of pipes, as strips, as tubes, as plates or in the form of mats.

29. The photoreactor as claimed in claim 22, wherein the LED plastic molded parts are provided in the form of pipes, as strips, as tubes, as plates or in the form of mats.

30. The photoreactor as claimed in claim 22, wherein the photoreactor contains a plurality of LED plastic molded parts, which are formed as tubes, pipes or plates, and these are combined as pipe, tube or plate bundles to form lighting inserts.

31. The photoreactor as claimed in claim 22, wherein the photoreactor is a photobioreactor containing LED plastic molded parts in tube or plate form, through which an alga suspension can flow, a plurality of tubes or plates being combined to form tube or plate bundles and forming a reactor unit through which a cooling medium flows in a shell space thereof.

32. The photoreactor as claimed in claim 22, wherein the LED plastic molded parts are based on crosslinked silicone rubbers, silicone hybrid polymers and/or silicone resins.

33. The photoreactor as claimed in claim 22, wherein the LED plastic molded parts contain a single silicone matrix of thermoplastic elastomers.

34. The photoreactor as claimed in claim 22, wherein the LED plastic molded parts contain a soft inner silicone matrix A that is enclosed by one or more harder silicone matrices B.

35. The photoreactor as claimed in claim 22, wherein the plastic matrix is provided with a topcoat C.

36. A method for carrying out a radiation-induced chemical reaction, comprising performing the reaction in a photoreactor as claimed in claim 22.

37. A method for producing alga biomass, comprising exposing an alga to light in a photoreactor as claimed in claim 22.

38. The method as claimed in claim 37, comprising autotrophic and heterotrophic production of valuable substances by means of the alga biomass.

39. A method for removing CO₂ from power plant or industrial waste gases, comprising feeding the CO₂ to a photoreactor as claimed in claim 22.

40. A method for producing hydrogen or other gaseous metabolic products, comprising exposing microalgae or microorganisms requiring light as an energy source and capable of forming the hydrogen or other gaseous metabolic products to light in a photoreactor as claimed in claim 22.

41. A method of generating energy or preparing a chemical, foodstuff, cosmetic or medical product, wherein the method comprises providing as a raw material an alga biomass produced in the photoreactor as claimed in claim 37.
